(19) United States
(12) Patent Application Publication
(10) Pub. No.: US 2007/0016122 A1
(43) Pub. Date: Jan. 18, 2007

(54) Orthopedic foot splint with an optional posterior strut and cuff

(76) Inventor: Gerald David Bowman, Winnetka, CA (US)

Correspondence Address: WHITE-WELKER & WELKER, LLC, P.O. BOX 199, CLEAR SPRING, MD, 21722-0199, US

(21) Appl. No.: 11/179,366

(22) Filed: Jul. 12, 2005

(51) Int. Cl. A61F 5/00; A61F 5/37

(52) U.S. Cl. 602/24; 602/23; 128/882

(57) ABSTRACT

A flexible orthopedic foot splint comprising two shoes containing screw receptacles in the soles; a bar contains one or more small apertures for connecting the shoes to the bar; two or more screws which fit through the apertures on the bar and attaches said bar to the shoes via said screw receptacles in the soles of said shoes; a posterior strut attachment that distally connects to the bar and one shoe via said screw receptacles; half circular cuffs attach to the strut attachment and wrap around an infants calf; said cuff is securely held to the leg via a hook and loop fastener wherein said bar is made of a low modulus plastic yielding great flexibility upon the application of force. With the orthopedic foot splint of the present invention, when a child attempts to stretch and pull his foot into plantarflexion and adduction the present invention allows some motion but limits excessive motion and then springs the foot back into the corrected alignment. As a result of the allowed motion the heel is not forced upwards in the shoe and the risk of the foot pulling out of the shoe diminished.

### FEDERALLY SPONSORED RESEARCH

Not Applicable

### SEQUENCE LISTING OR PROGRAM

Not Applicable

### CROSS REFERENCE TO RELATED APPLICATIONS

Not Applicable

### TECHNICAL FIELD OF THE INVENTION

The present invention relates generally to orthotics and prosthetics. More specifically the present invention relates to an orthopedic foot splint and optional posterior strut that offers better control of plantarflexion and internal rotation or one or both feet.

### BACKGROUND OF THE INVENTION

Congenital deformities of the limbs occur in a small percentage of the population. The most common deformity is clubfoot AKA talipes equino varus (TEV), affecting approximately one in 750 babies. TEV deformity results in a plantarflexed, internally rotated and adducted tibia, ankle and foot complex. Treatment options involve any combination of surgical realignment, casting, taping and external splinting.

The deformity involves internal rotation of the leg and foot, plantar flexion (equinus) of the foot, inversion (turning in) of the hindfoot, and adduction (turning under) of the forefoot and midfoot giving the foot a kidney-shape.

General accepted concepts of treatment have always been to manipulate the feet and ankles into a corrected alignment and then rigidly hold the feet and ankles in a position opposite of the deformity for an extended period of time, for example: external rotation, eversion, abduction and dorsiflexion. Bracing, when used, is usually continued until the child reaches the age of 3 to 4.

The Dennis Browne Bar (also known as Dennis Browne Splint, Dennis Browne Splint, Dennis Browne Bar, Abduction Bar) is a brace that has been in common use for over 60 years and remains the most widely used splinting/bracing option. The original prior art was invented before the advent of plastics therefore metal was the only material option.

Generally accepted principles of treatment teach that the foot and ankle must be maintained in a rigid position to maintain the correction and therefore metal with its high modulus of rigidity has always been considered to be the material of choice and there has never been reason to question this choice.

Compliance with bracing can be very problematic, particularly in children over 2 years old and often results in discontinuation with bracing and therefore increased risk of recurrence. Failure to keep the child consistently in their brace is cited as being the most common cause of recurrence of the deformity and necessitates further surgery and/or castings.

There are several well-known problems that occur with Dennis Browne type splinting/bracing, such as: keeping the shoes attached to an infant's feet; heel sores resulting from the heel rubbing against the back of the shoe; poor control of plantarflexion (equinus), difficulty changing the infants clothes, children over 18 months of age often refuse to wear shoes/bar, injuries to infant/parent or damage to the infants crib and a delay in motor development. Therefore it is an objection of the present invention to teach a brace that overcomes the aforementioned shortcomings in the prior art.

It is hypothesized that keeping the shoes attached to an infant's feet; heel sores resulting from the heel rubbing against the back of the shoe, and poor control of plantarflexion (equinus) occur as a result of the attempt by the infant to move his/her feet. As the infant stretches, the feet attempt to plantarflex and adduct. As the metal Dennis Browne Bar prevents this motion, there is a resulting upwards-directed force placed upon the heel of the foot causing the heel to attempt to lift up and out of the shoe.

Many infants have feet whereby the posterior shape is very rounded. It is therefore very difficult for the shoe to apply a counteractive downwardly directed force to block the heels from lifting up in the shoe. Infants with such feet are very prone to pulling out of their shoes. It is therefore an objective of the present invention to teach a brace that eliminates this shortcoming.

Consistent motions of the heel pulling up as the infant stretches can result in friction sores on the back of the heel. It is therefore an objective of the present invention to teach a brace that eliminates this shortcoming.

With the traditional Dennis Browne Bar, the generally agreed solution for plantarflexion control is to apply a bend to the metal bar. However this bend in the bar does not position the foot in more dorsiflexion, it actually places a valgus thrust on the ankles, which results in a valgus and flexion moment to the knees that may encourage some mild dorsiflexion. It is a biomechanically ineffective protocol. It is therefore an objective of the present invention to teach a brace that eliminates this shortcoming.

For bracing to be effective it needs to be worn all the time. As the prior art rigidly attaches both feet to each other, changing an infant's clothes necessitates that the apparatus be completely removed. This is time consuming for the parent, negates the effect of the brace and tempts the parent to leave the infant out of the brace for lengths of time. It is therefore an objective of the present invention to teach a brace that eliminates this shortcoming.

As children get older, it becomes increasingly hard to keep them in rigid bracing systems. Many older children create such a commotion over wearing the splints that parents' wills are broken and the braces become unused. It is therefore an objective of the present invention to teach a brace that eliminates this shortcoming.

Metal Dennis Browne bars are rigid and have been known to cause injury to infants and their parents and cause damage to the infant's crib. It is therefore an objective of the present invention to teach a brace that eliminates this shortcoming.

Infants wear the brace during the period of locomotor development whereby a child learns to crawl and pull to standing. These activities are difficult for an infant to achieve while wearing a rigid bracing system. The metal bar has two external bolts that become the weight bearing points on attempting to stand, thereby reducing the area of contact with the ground and making these activities increasingly difficult. It is therefore an objective of the present invention to teach a brace that eliminates this shortcoming.

Others have recognized the aforementioned problems with fixed position splinting and the need for improved compliance. The prior art mentioned describes modifications to the Dennis Brown configuration to include hinges that allow for free motion in certain directions.

More specifically, U.S. Pat. No. 4,606,334 issued to Salmon teaches a pair of brackets for attachment to the feet which are connected by a number of links pivotably attached to the shoe brackets which are maintained in a constant parallel spaced apart relation preventing transverse movement. U.S. Pat. No. 4,520,803 issued to Quest teaches the use of a longitudinally extending splint bridge. U.S. Pat. No. 4,412,536 issued to Kurtz, et al. teaches the use of linkage comprising two pairs of parallelogram links to control foot movement. U.S. Pat. No. 4,303,065 issued to Ericson and U.S. Pat. No. 4,249,523 issued to Bidwell both teach alternative hinge systems for restricting foot movement.

Unfortunately, these devices have achieved very little acceptance in the field, due to their higher cost, and inferior results due to them allowing too much motion thereby affecting the ability of the device to maintain stretch to the soft tissues and hence prevent recurrence. It is therefore an objective of the present invention to teach a brace that eliminates these shortcomings.

Others have recognized the need for a flexible system. For example, U.S. Pat. No. 4,040,416 issued to Zentman describes a spring steel abduction bar of the same design as the Dennis Browne bar but made of a spring steel material to allow motion. This splint however still fails to address the plantarflexion problem.

What is needed is a splinting design that will rigidly maintain rotation and strongly encourage dorsiflexion and abduction, that will be easy to apply, remain applied for long periods, prevent heel sores and minimize the disruption to the child's natural locomotor development.

### SUMMARY OF THE INVENTION

The flexible bracing system disclosed by the present invention is a flexible bracing system that exhibits significantly different properties from the rigid Dennis Browne splint and alternative designs previously discussed. The addition of a posterior strut and cuff makes the bracing system more effective in controlling plantarflexion and internal rotation than any of the prior art. The addition of a clasp in the connecting bar allows for easier clothing change and is a more user-friendly design that will encourage greater number of hours in the brace and therefore make the bracing system more effective.

In accordance with the present invention an Orthopedic Foot Splint with an Optional Posterior Strut and Cuff that overcomes the aforementioned shortcomings and problems of the prior art is now disclosed.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 illustrates the Dennis Browne Bar, the most widely used splinting/bracing option known in the prior art;

FIG. 2 illustrates a perspective view of a universal splint design known in the prior art;

FIG. 3 illustrates a perspective view of a the orthopedic foot splint with an optional posterior strut and cuff of the present invention;

FIG. 4 is an exploded view of the orthopedic foot splint of the present invention including a separation clasp;

FIG. 5 is a perspective view of the orthopedic foot splint of the present invention including a separation clasp.

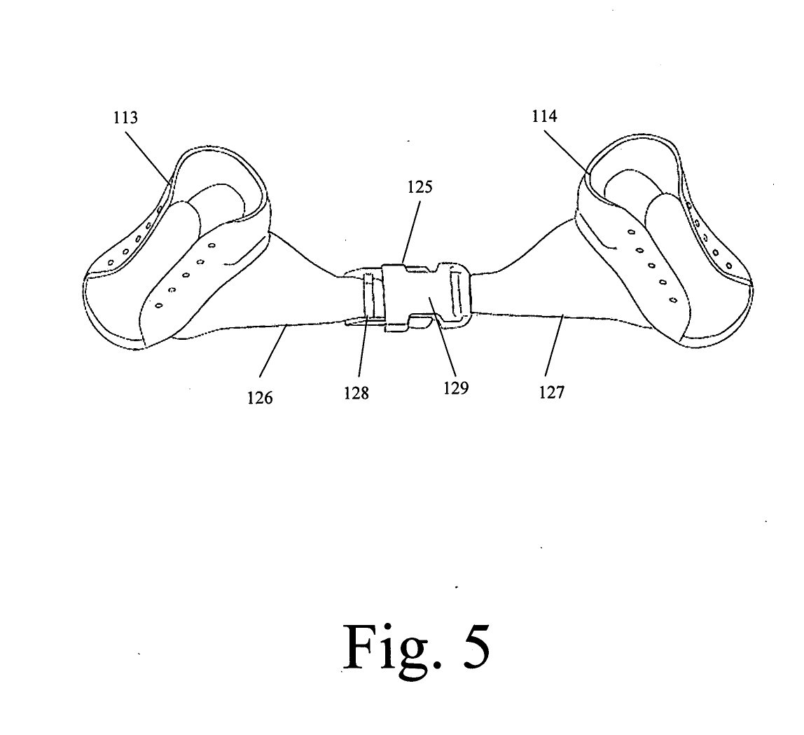

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized and logical, mechanical, electrical, and other changes may be made without departing from the scope of the present invention. The following detailed description is therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention.

Referring to the figures, it is possible to see the various major elements constituting the apparatus of the present invention. The invention is an Orthopedic Foot Splint with an Optional Posterior Strut and Cuff.

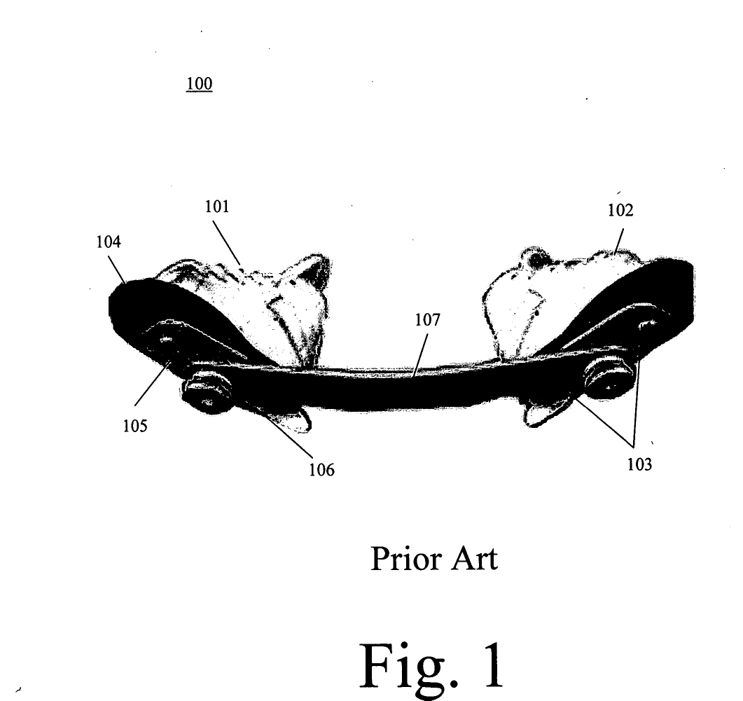

Now referring to FIG. 1, the Dennis Browne Bar 100 is illustrated. It consists of two open toe shoes 101 and 102 that have screw receptacles on the soles 104. Metal brackets 105 are attached to the shoes 101 and 102 via two screws 103 that lock into the screw receptacles. A serrated edged 106 disc is attached to the metal bracket 105. There is a steel or aluminum bar 107 with a circular aperture at either end. The aperture has a serrated edge and connects to both shoes 101 and 102 via the serrated discs on the brackets 105. The serrations allow for placement of the two shoes/brackets at varying degrees of rotation in respect to the metal bar 107.

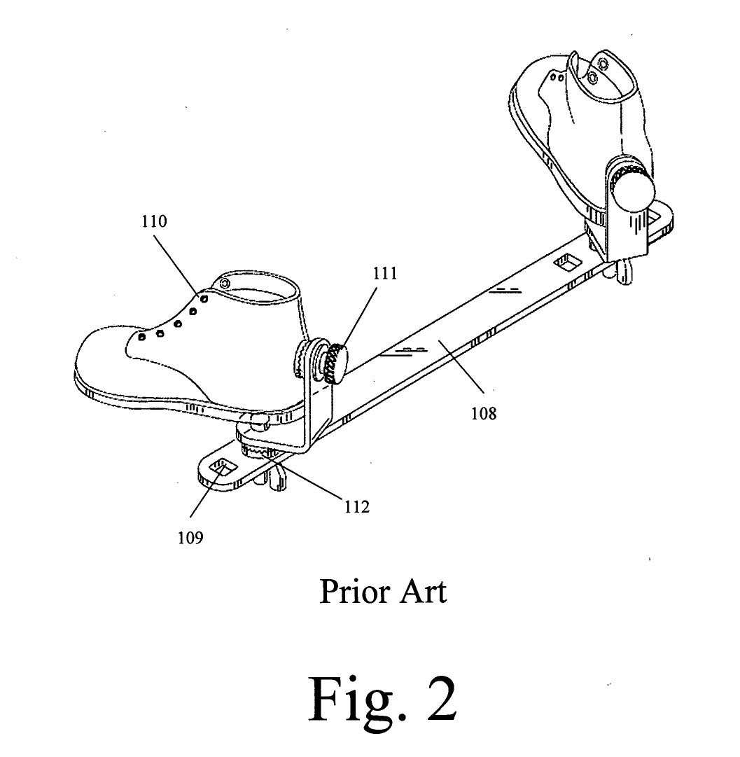

Now referring to FIG. 2, a perspective view of a universal splint design known in the prior art is illustrated. The typical splint design currently used is an assembly comprised of a conventional child's shoe 110 with one or more attachment elements 111 such as serrated disks or equivalent attachment means. A bar 108 with a plurality of apertures 109 at either end and one or more attachment elements 112 such as serrated disks or equivalent attachment means.

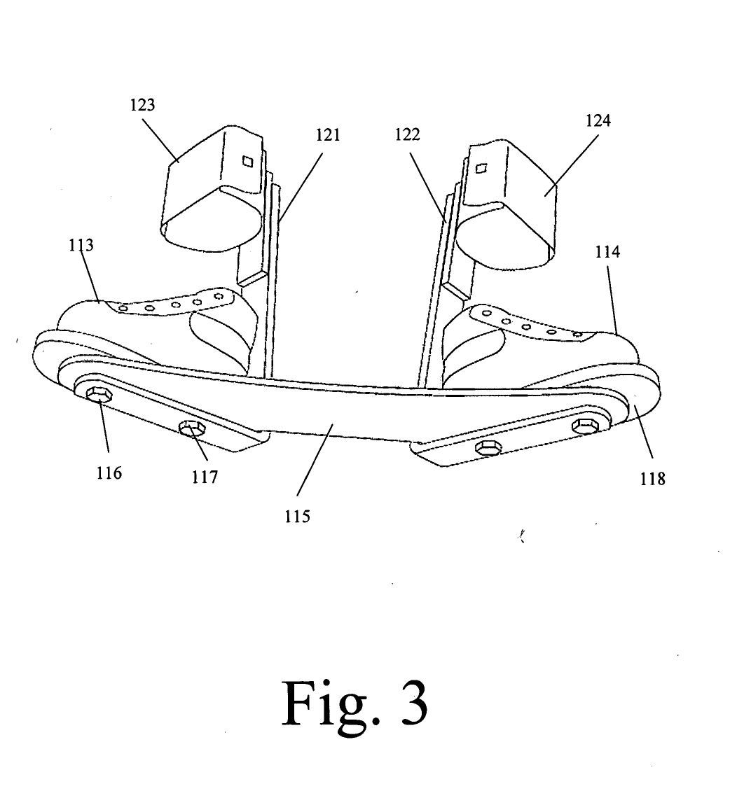

The present invention, as illustrated in FIG. 3, is comprised of two shoes 113 and 114 of the same description as those specified in the prior art. The shoes 113 and 114 contain screw receptacles in the soles. Attached to these screw receptacles is a plastic bar 115 connecting the shoes 113 and 114. The bar can be made from polypropylene and polyethylene or other similar plastic material. The plastic bar 115 consists of a length between 8 and 14 inches that widens at both ends and contains several small apertures 119 and 120. Two screws 116 and 117 fit through the apertures on the plastic bar 115 and attach the plastic bar 115 to the shoes 113 and 114 via the screw receptacles in the soles 118 of the shoes 113 and 114. The shoes 113 and 114 can be positioned in different degrees of rotation depending upon which apertures 119 and 120 are used on the plastic bar 115.

The bar 115 is made of plastic and hence has significantly different properties than the metal bars previously used in the prior art. The plastics used in the present invention have a relatively low modulus of rigidity and as a result, the bar flexes easily upon the application of force. However the resiliency and spring in the material forces the feet to return to the desired position after relaxation of the muscles. As a result, instead of a rigid bracing system the present invention is a flexible bracing system overcoming the shortcomings previously mentioned in the prior art.

With the flexible bracing system, as the child attempts to stretch and pull his foot into plantarflexion and adduction the present invention allows some motion but limits excessive motion and then springs the foot back into the corrected alignment. As a result of the allowed motion the heel is not forced upwards in the shoe and the risk of the foot pulling out of the shoe diminished.

Previous understanding would expect that a flexible bracing system would be ineffective as it would prevent the stretch to the soft tissues from being maintained and result in recurrence of the deformities. However, as the spring of the plastic material acts to return the foot and leg to the desired position, the majority of time is spent in a corrected alignment. Preliminary results indicate that the system is more effective at preventing the shoes from coming off, creating less pressure sores, and appears to be effective in maintaining correction and could prove to be superior due to the increase in the number of hours the shoes remain on the feet.

Patients with TEV require plantarflexion control. With the traditional DB splint, the generally agreed treatment is to apply a bend to the metal bar. However, this bend in the bar does not position the foot in more dorsiflexion, it actually places a valgus thrust on the ankles which results in a valgus and flexion moment to the knees that may encourage some mild dorsiflexion. It is a biomechanically ineffective protocol.

The present invention consists of posterior strut attachments 121 and 122 that distally connects to the abduction bar 115 and shoes 113 and 114 via the screw receptacles. Proximally, plastic half circular cuffs 123 and 124 attach to the strut attachments 121 and 122 and wrap around an infants calf. The cuffs 123 and 124 are securely held to the leg via a hook and loop fastener. The posterior strut attachments 121 and 122 can be applied to the left leg/foot, right leg/foot or both legs/feet.

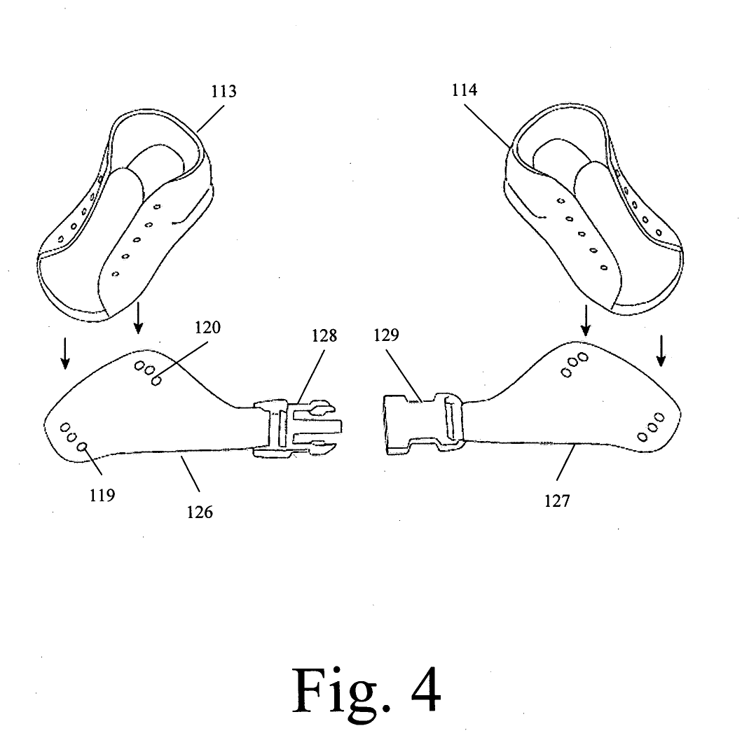

Now referring to FIG. 4, an additional option with the flexible bracing system is the use of a separation clasp 125 in the horizontal bar 115 to allow for easy separation of the feet/shoes for easier donning/doffing of clothes. The horizontal bar 115 now becomes two separate bars 126 and 127 that connect via an easy to use clasp 125. Connection of the two bars 126 and 127 requires simply pushing the two sections 128 and 129 together. Separation of the two bars 126 and 127 requires simple thumb and finger pressure on the sides of the clasp 115.

It is appreciated that the optimum dimensional relationships for the parts of the invention, to include variation in size, materials, shape, form, function, and manner of operation, assembly and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. An orthopedic foot splint comprising:
two shoes containing screw receptacles in the soles;
a bar contains one or more small apertures for connecting the shoes to the bar;
two or more screws which fit through the apertures on the bar and attaches said bar to the shoes via said screw receptacles in the soles of said shoes;
a posterior strut attachment that distally connects to the bar and one shoe via said screw receptacles;
proximally, half circular cuffs attach to the strut attachment and wrap around an infants calf;
said cuff is securely held to the leg via a hook and loop fastener.

2. The orthopedic foot splint of claim 1 wherein the bar can be made from polypropylene and polyethylene or other plastic material.

3. The orthopedic foot splint of claim 1 wherein bar consists of a length between 8 inches and 14 inches.

4. The orthopedic foot splint of claim 1 wherein said shoes can be positioned in different degrees of rotation depending upon which apertures are used on the bar to connect said shoes with said bar.

5. The orthopedic foot splint of claim 1 wherein the posterior strut attachment can be applied to the left leg/foot or the right leg/foot.

6. The orthopedic foot splint of claim 1 wherein two posterior strut attachments can be applied, one to the left leg/foot and one to the right leg/foot.

7. The orthopedic foot splint of claim 1 further comprising a separation clasp in the bar as means to allow for easy separation of the feet/shoes, wherein said bar now becomes two separate bars that connect said separation clasps.

8. A flexible orthopedic foot splint comprising two shoes containing screw receptacles in the soles; a bar contains one or more small apertures for connecting the shoes to the bar; two or more screws which fit through the apertures on the bar and attaches said bar to the shoes via said screw receptacles in the soles of said shoes; a posterior strut attachment that distally connects to the bar and one shoe via said screw receptacles; half circular cuffs attach to the strut attachment and wrap around an infants calf; said cuff is securely held to the leg via a hook and loop fastener wherein said bar is made of a low modulus plastic yielding great flexibility upon the application of force.
